(19) United States
(12) Patent Application Publication
(10) Pub. No.: US 2022/0168153 A1
(43) Pub. Date: Jun. 2, 2022

(54) PROCESS FOR MAKING A COMPOSITE ELASTIC TAPE, ELASTIC TAPE SO OBTAINED AND PANTY DIAPER COMPRISING SAID ELASTIC TAPE

(71) Applicant: DELTA SRL UNIPERSONALE, FIESCO (CR) (IT)

(72) Inventor: Angelo GHIDELLI, FIESCO (CR) (IT)

(21) Appl. No.: 17/441,086

(22) PCT Filed: Mar. 6, 2020
(86) PCT No.: PCT/IT2020/050052

§ 371 Date: Sep. 20, 2021

(30) Foreign Application Priority Data
Mar. 21, 2019 (IT) 102019000004133

(51) Int. Cl. A61F 13/15; B32B 3/26; B32B 38/00; B32B 5/04; B32B 5/12

(52) U.S. Cl. CPC A61F 13/15593; A61F 13/15699; A61F 13/15756; B32B 2038/045; B32B 38/1875; B32B 5/04; B32B 5/12; B32B 3/26

(57) ABSTRACT

Disclosed is a process for making a composite elastic tape, elastically extendable along its longitudinal direction, including: providing a tape of elastic material having a main elongation direction; providing first and second tapes of fibrous and porous material; cutting the tape of elastic material into strips along the direction; tensioning the strips of elastic material along the direction to elastically lengthen and distance them; arranging the first and the second tape of fibrous and porous material respectively above and below the tensioned, elastically elongated and spaced strips; and fixing the strips while being tensioned, elastically elongated and spaced, between the first and the second tape of fibrous and porous material in an undeformed condition, to make a composite elastic material shaped like a tape. Also disclosed is the composite elastic tape thus obtained, suitable for the waist region of a diaper as well as other products incorporating such tape.

### BACKGROUND OF THE INVENTION

### Field of the Invention

The present invention relates to the field of hygienic-sanitary articles and relates to a process for making a composite elastic tape, elastically extendable along the longitudinal direction of the tape itself.

The present invention also relates to the composite elastic tape thus obtained, intended to be applied in the waist region of a wearable diaper of the pull-on type (or panties), indifferently for children or adults, as well as the diaper incorporating said elastic tape.

However, the scope of the invention is not limited only to this possible field of application.

### Description of the Related Art

In the field of hygienic sanitary articles that can be worn in the form of panties, diapers are known which comprise a central body intended to be arranged according to a “concave” configuration around the crotch area of the user.

Said central body includes the actual absorbent core.

In order to allow the diaper to be worn in a stable and comfortable way by the user, said diaper comprises a ring made with an elastic tape, associated with said central body, for example by welding, which is capable of encircling the waist of the user.

This elastic tape is of the composite type, made by coupling two outer layers of fibrous material, for example TNT, and an intermediate layer of elastic material.

In order to be really comfortable for the user, said elastic tape must perform both a structural function, encircling the user's waist and ensuring the necessary degree of wearability, and allowing perspiration, avoiding unwanted maceration of the user's skin due to local stagnation of humidity.

It is therefore essential that this elastic tape is breathable.

From U.S. Pat. No. 6,994,761 B2 a method is known for forming a breathable elastic tape applicable to a disposable absorbent garment, and therefore to a diaper.

Said method substantially comprises the steps of:
selecting a first and a second layer of fibrous material;
selecting an elastic polymeric material for a layer of extensible material;
positioning the layer of extensible material between the first and second layers of material to form a set of layers;
identifying one or more bonding points on the set of layers for their coupling;
fastening together the first layer, the second layer and the intermediate layer of extensible material by ultrasonic junction of the first and second layers in one or more junction points, at which openings are formed which also affect the elastic extensible material so as to create actual perspiration holes.

The elastic tape obtained is therefore configured as a trilaminate, where each layer of material overlapped has the same width with respect to the longitudinal direction of development and comprises along its entire surface transpiration holes which affect all three overlapping layers.

As for the coupling methods between the layers, the technique of ultrasonic welding is known from document U.S. Pat. No. 6,994,761 B2, while known more traditional techniques provide for the simple gluing of the layers, which would then require subsequent steps of drilling the thus obtained trilaminate.

Current production lines that use the method described above exhibit process difficulties related above all to the coupling and joining steps of the layers.

For example, the ultrasonic joint is disadvantageously characterized by the possibility of damaging layers with thin thicknesses, or with thin sections, especially for the layers of fibrous material that make up the outer layers of the composite tape.

Furthermore, the ultrasonic joint requires a careful study of the welding joint, and in particular in the subject application, of the exact welding point which must also have the function of air passage.

Even more disadvantageously, with the processing methods described above, large quantities of elastic material are used, which instead could be optimized, if better distributed within the tape.

A further drawback consists in the reduced breathability of the elastic tape obtained, being the elastic material, which occupies the entire width of the strip, totally impermeable to air and therefore the function of perspiration entrusted only to the holes obtained from the ultrasound junction points.

### SUMMARY OF THE INVENTION

The invention aims to overcome these limits by defining an efficient and fast process for making a composite elastic tape which optimizes production without wasting material.

It is also an object of the invention to provide a composite elastic tape to be used on panty type diapers which ensures maximum wearability, high breathability of the product and the best comfort for the user.

These objects are achieved with a process for manufacturing a composite elastic tape as disclosed and claimed.

Further preferred steps of the process according to the invention are also disclosed and claimed.

The subject of the invention also includes a composite elastic tape obtained with this process and a panty type diaper which uses this composite elastic tape.

The process object of the present invention, and the product obtained with it, offer important advantages compared to the traditional production methods and to the products thus obtained and used for making diapers of the panty type.

The process according to the invention allows making a highly breathable elastic composite tape even without the use of ultrasonic welding for its perforation, since it is not trilaminate, that is, it is not composed of three layers of equal width.

The breathability function is directly entrusted to the layers of fibrous material that make up the external walls of the elastic composite tape: the fibrous material that is already breathable, which advantageously is not entirely covered by the air-impermeable elastic material, ensures this important function.

The process implemented creates breathability in the non-elasticized areas: the fact of keeping the strips of elastic material spaced apart leaves in fact portions of fibrous material free and suitable for the passage of air.

Furthermore, the strips of elastic material obtained by cutting do not necessarily have to be mechanically moved away from each other: the step of elongation in their direction of longitudinal development thins them and therefore distances them.

Alternatively, if, for constructional reasons, the strips are to be spaced apart from each other, a mechanical adjustment of their position could occur, for example by means of a roller system according to the prior art.

The disclosed process operates continuously, quickly and efficiently, with a considerable increase in the production of tape and final product.

### BRIEF DESCRIPTION OF THE DRAWINGS

Further features and advantages of the invention shall appear more clearly from the following description of a preferred embodiment, made by way of an indicative and non-limiting example with reference to the figures, in which:

FIG. 1 is a schematic representation of a system which carries out a process for manufacturing a composite elastic tape according to the invention;

FIG. 2 is a schematic representation of the composite elastic tape according to the invention, ideally divided into the different construction steps of the process of FIG. 1;

FIG. 3 is a simplified top plan view of a portion of composite elastic tape according to the invention;

FIGS. 4 and 5 show, in a simplified axonometric view, a diaper of the panty type comprising the composite elastic tape of FIG. 3, and a detail thereof, respectively.

### DESCRIPTION OF THE PREFERRED EMBODIMENTS

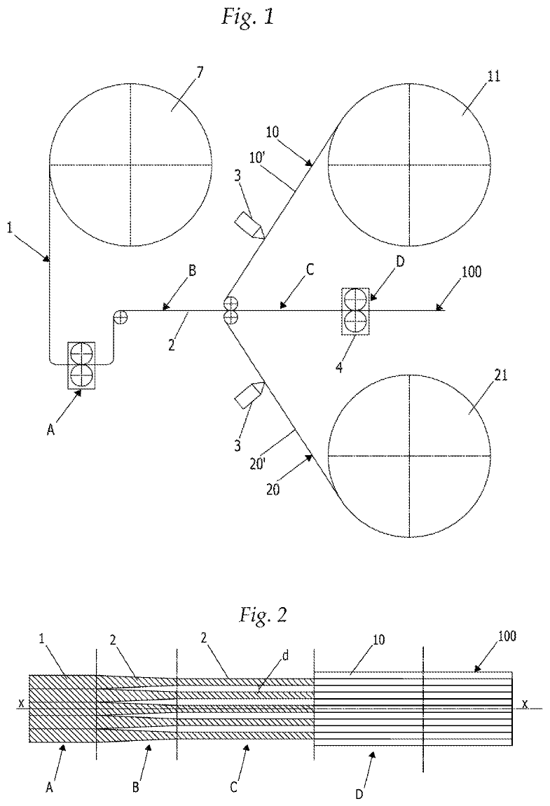

The steps of the process for manufacturing a composite elastic tape 100 according to the invention are described below, with reference to the system diagram illustrated in FIG. 1.

The process essentially comprises the steps of:
unwinding, from a first reel 7, a web 1 of elastic material having a main elongation direction x;
longitudinal cutting of said web 1 of elastic material into strips 2 along said direction x;
tensioning and elongation of said strips 2 of elastic material along said direction x, according to the degree of force preferred or imposed by the regulations;
unwinding, from a second reel 11, a first web 10 of fibrous and porous material, for example TNT, above said strips 2;
unwinding, from a third reel 21, a second web 20 of fibrous and porous material, for example TNT, below said strips 2;
coupling said tensioned strips 2, elastically elongated and spaced, of elastic material with said first 10 and second 20 strips of fibrous and porous material kept in an undeformed state, to obtain a composite elastic material shaped like a tape 100, substantially having the width of said tapes 10, 20 of TNT.

In order to carry out said coupling, said process comprises, in the illustrated preferred variant, the step of spraying glue 3 on the sides 10′, 20′ of said first 10 and second 20 tape of fibrous and porous material facing said elongated strips 2 before being arranged above and below them.

Said glue 3 could be sprayed on the whole surface of the sides 10′, 20′, or, more advantageously, it could be sprayed locally along only the portions of tape of fibrous and porous material which will correspond, in contact, with the strips 2 of elastic material.

Alternatively, the glue could be sprayed directly on the sides of said tensioned strips 2, elastically elongated and spaced, before being coupled with said first and second layers of fibrous and porous material. In this way, there would be an advantageous saving on the quantity of glue used.

To increase said coupling between the layers, or as an alternative to the aforementioned spraying step of glue 3, said process comprises the step of passing said elastic composite material shaped as a tape 100 between heat-sealing rollers 4.

The design of this heat sealing may be of various types and decorations to meet the market demands on the finished product that will then be made, namely the diaper.

It is clear that in more simplified alternative variants, the coupling of the layers may also take place by means of gluing only or hot welding only with heat-sealing rollers.

FIG. 2 specifically illustrates the process steps with particular attention to what happens to the elastic material, identifying four process areas:
a zone A for longitudinally cutting the strips of elastic material 1 into strips 2;
a zone B for tensioning and elongating said strips 2;
an overlapping zone C of said strips 2 with the two tapes 10, 20 of fibrous and porous material;
a zone D for welding the layers to obtain the composite elastic tape 100.

As is clear from the diagram in FIG. 2, the tensioning and elongation step of the strips 2 of elastic material along said direction x automatically and advantageously causes their thinning and spacing.

At the moment of the coupling step with the tapes 10, 20 of fibrous material, said strips 2 are detached from each other by a suitably defined distance “d”.

By way of example only, said strips 2 of elastic material may be cut with a width of between 5 mm and 100 mm, and the distance “d” left between said strips 2 of elastic material when elongated may have a size of between 5 mm and 60 mm.

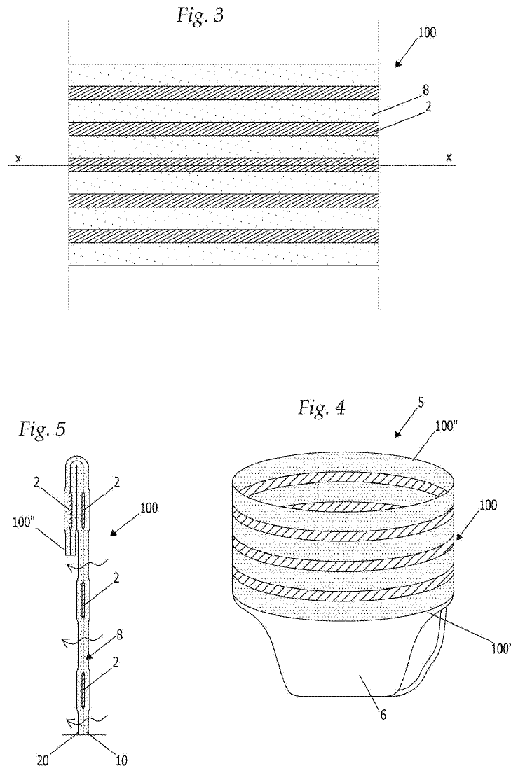

After gluing and/or welding, the composite elastic tape 100 appears itself as an alternation, in the transverse direction to the x direction, of elastic strips 2 of composite material and bands 8 of only superimposed porous and breathable fibrous material (FIG. 3).

Air flows through these bands 8, which is essential to make the tape 100 breathable and ensure maximum comfort to the diaper on which it will be fixed.

Upon release of the elastic strips 2, the composite elastic tape 100 retracts and curls to be able to lengthen in use.

With reference to FIG. 4, a diaper 5 is shown comprising an absorbent central body 6 and a composite elastic tape 100 closed on itself in a ring and fixed, along a first edge thereof 100′, to said absorbent central body 6.

Said ring-closed composite elastic tape 100 is adapted to encircle the waist of the user, be it a child or an adult, so as to make said diaper 5 wearable as a panty.

With particular reference to FIG. 5, a detail of a second free edge 100″ of said composite elastic tape 100, distal to the absorbent central body 6 and substantially acting as a belt area, is illustrated.

To increase, and at least double, the elastic holding force, and therefore the force exerted by the material at the user's waist, said composite elastic tape 100 is partially folded on itself, so as to overlap at least two of said strips 2 of elastic material.

The fixing of said second edge 100″ in a folded configuration on itself still takes place by means of a heat welding step, before the ring closure and the application of the tape 100 to the absorbent central body 6 of the diaper 5.

1. Process for making a composite elastic tape comprising the steps of:
providing a tape of elastic material having a main elongation direction;
providing a first and a second tape of fibrous and porous material, of the process comprising:
cutting said tape of elastic material into strips along said direction;
tensioning said strips of elastic material along said direction so as to elastically lengthen them and distance them;
arranging said first and said second tape of fibrous and porous material respectively above and below said tensioned, elastically elongated and spaced strips;
fixing said strips while being tensioned, elastically elongated and spaced, between said first and said second tape of fibrous and porous material in an undeformed condition, to make a composite elastic material shaped like a tape.

2. The process for making a composite elastic tape according to claim 1, wherein said step of cutting said tape of elastic material comprises the step of cutting strips with a width of between 5 mm and 100 mm.

3. The process for making a composite elastic tape according to claim 1, wherein the step of spacing said strips comprises the step of spacing them with a distance of between 5 mm and 60 mm.

4. The process for making a composite elastic tape according to claim 1, wherein said step of fixing said tensioned, elastically elongated and spaced strips between said first and said second fibrous and porous material comprises the step of spraying glue, alternatively on the sides of said first and second tape of fibrous and porous material facing towards said tensioned, elongated and spaced strips, before being arranged above and below them, or on the sides of said tensioned, elastically elongated and spaced strips before being coupled with said first and second layers of fibrous and porous material.

5. The process for making a composite elastic tape according to claim 1, wherein said step of fixing said strips while they are tensioned, elastically elongated and spaced, between said first and said second tape of fibrous and porous material comprises the step of passing said composite elastic material shaped like a tape into heat-sealing rollers.

6. Composite elastic tape that is made by the process according to claim 1.

7. Panty diaper comprising an absorbent central body, comprising the composite elastic tape according to claim 6, wherein said composite elastic tape is closed on itself as a ring and fixed along a first edge to said absorbent central body, and is adapted to surround the waist of a user so as to make said diaper wearable as a panty.

8. The panty diaper according to claim 7, wherein said composite elastic tape is partially folded on itself along a second edge distal to said absorbent central body, so as to superpose at least two of said strips of elastic material, increasing and at least doubling the elastic holding force at the user's waist.

9. The process for making a composite elastic tape according to claim 4, wherein said step of fixing said strips while they are tensioned, elastically elongated and spaced, between said first and said second tape of fibrous and porous material comprises the step of passing said composite elastic material shaped like a tape into heat-sealing rollers.

10. Composite elastic tape that is made by the process according to claim 2.

11. Composite elastic tape that is made by the process according to claim 3.

12. Composite elastic tape that is made by the process according to claim 4.

13. Composite elastic tape that is made by the process according to claim 5.

14. Composite elastic tape that is made by the process according to claim 6.
